(19) United States
(12) Patent Application Publication
(10) Pub. No.: US 2025/0280940 A1
(43) Pub. Date: Sep. 11, 2025

(54) COSMETIC PRODUCT WITH SURFACE DECORATION OF DYNAMIC IMAGES

(71) Applicant: INTERCOS S.p.A., Milano (IT)

(72) Inventors: Giuseppe LATTANZI, LIMBIATE (MB) (IT); Giovanni VENTURELLI, PADERNO DUGNANO (MI) (IT)

(21) Appl. No.: 18/282,137

(22) PCT Filed: Jun. 21, 2021
(86) PCT No.: PCT/IB2021/055445

§ 371 Date: Sep. 14, 2023

(30) Foreign Application Priority Data
Mar. 15, 2021 (IT) 102021000006122

(51) Int. Cl. A45D 33/18; A45D 33/00; A45D 40/00; A61K 8/02; A61Q 1/06

(52) U.S. Cl. CPC A45D 33/18; A45D 33/008; A45D 40/00; A61K 8/0237; A61Q 1/06; A45D 2040/0012; A45D 2040/0025

(57) ABSTRACT

A powder cosmetic product in the form of a flat wafer or plate or extruded rod for lipsticks includes a cosmetic body with a visible surface provided with a plurality of protruding stripes with triangular section. Each stripe has a vertex, a first inclined side decorated with a portion of a first image, and a second inclined side decorated with a portion of a second image. By observing the cosmetic body from an observation point (A) angled so that only the first inclined side of each stripe can be seen, only the first image is visible, while by observing the same cosmetic body from an observation point (B) angled so that only the second inclined side of each stripe can be seen, only the second image is visible.

### BACKGROUND OF THE INVENTION

The present invention relates to a cosmetic product having a surface decoration consisting of images with a dynamic effect, this term meaning the capacity to show separate images which can vary with the angle of the observation point.

The cosmetic product can be made of pressed, melted, baked powder or it can be in the form of an extruded rod.

The current surface decoration techniques of a cosmetic product allow creating “static” images, i.e., visible in the same manner from any observation point, on the surface thereof.

JP 2005-313129 A discloses the cover body of a compact container in which a base material has a sight surface provided with a plurality of protruding stripes with triangular section. Each stripe has a vertex, a first inclined side decorated with a portion of a first color and a second inclined side decorated with a second color.

### SUMMARY OF THE INVENTION

It was the object of the present invention to develop a technique for forming and decorating the surface of a cosmetic product which would give rise to two or more surface images which can be seen separately as a function of the observation point angle.

The aforesaid object is achieved by a cosmetic product made of pressed, or melted and baked, or extruded powder or in the form of a rod or stick, such as a lipstick, having a body with a visible surface consisting of a plurality of protruding stripes with triangular section, each having an upper vertex, a first inclined side decorated with a portion of a first image, and a second inclined side decorated with a portion of a second image.

The shaping of the surface of the cosmetic product, with stripes with triangular section, allows observing the same surface from two different observation points angled so as to make respective sides of the stripes visible without being able to notice the opposite sides.

Thereby, it is possible to decorate the same cosmetic product with two different images which are separately visible from two different observation points, thus obtaining the so-called “lenticular effect”.

The inclination of the sides of the stripes, their width and their height must comply with proportion rules, which also depend on the cosmetic product to which the “lenticular” effect is intended to be applied. In particular, the inclination of the sides (or sidewalls) of the stripes must be from 40° to 60°, the base width from 0.5 mm to 3.0 mm, and the height from 0.2 mm to 1.5 mm.

### BRIEF DESCRIPTION OF THE DRAWINGS

The features of the present invention will be better understood from the following detailed description of exemplary embodiments shown in the accompanying drawings, in which:

FIG. 1 is a plan view of a cosmetic product with parallel surface stripes and a decoration with a double image according to the present invention;

FIG. 2 is a sectional view according to line II-II in FIG. 1;

FIG. 3 is an enlarged perspective depiction of a surface stripe of the cosmetic body in FIGS. 1 and 2;

FIGS. 4 and 5 show views of the decorated surface of the cosmetic product from observation points with opposing angles as shown in FIG. 2;

FIG. 6 shows a front view of a box-like package including the cosmetic product in FIG. 1;

FIG. 7 shows a front view of another example of a package including a cosmetic product according to the present invention; and

FIG. 8 shows a lipstick applicator including a lipstick rod with the decorative features of the present invention.

### DETAILED DESCRIPTION OF THE INVENTION

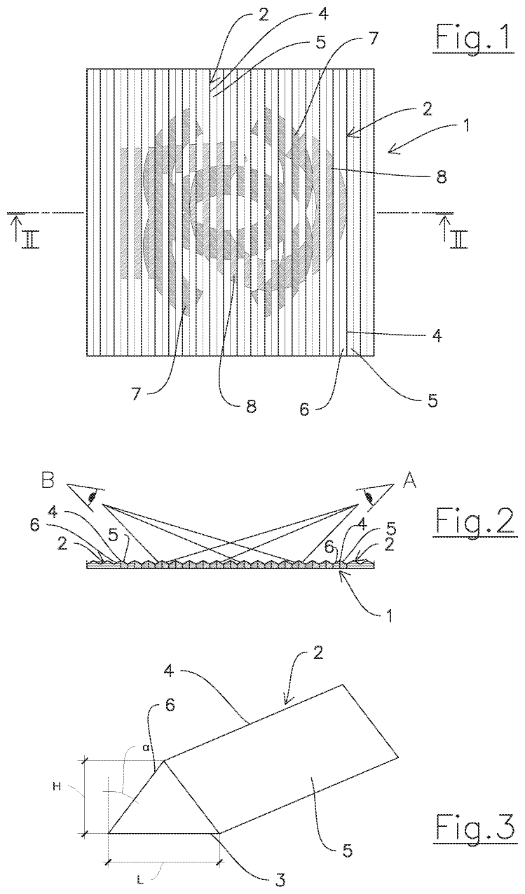

The cosmetic product in FIGS. 1 and 2 has a square flat plate-or wafer-shaped body 1 made of pressed, or melted and baked, or extruded cosmetic powder, which has an upper surface shaped so as to have a plurality of protruding stripes 2, by way of example parallel, such as that shown on an enlarged scale in FIG. 3. Obviously, the shape of the plate is completely indifferent, since it could be rectangular, polygonal, circular, elliptical, or any other desired shape without this affecting the scope of the invention.

As indicated in FIG. 3, each stripe 2 has a triangular section with a base 3 of width L, an upper vertex 4 of height H, and inclined lateral sides 5 and 6 with inclination a.

Width L, height H and inclination a of the stripes 2 must comply with proportion rules also depending on the cosmetic product used. In particular, the inclination a must range from 40° to 60°, the base width L must range from 0.5 mm to 3.0 mm, and the height H from 0.2 mm to 1.5 mm.

As shown in FIG. 1, the lateral sides 5 of the triangular stripes 2 are decorated with portions of a first image 7, while the lateral sides 6 of the same triangular stripes are decorated with portions of a second image 8.

The decoration of the surface of the cosmetic product can be obtained by various methods, even as a function of the type of cosmetic product. The following methods can be indicated by way of example:
ink-jet—four-color printing by means of a cosmetic-type ink jet printer;
spray+laser—total covering of the surface with a pearlescent spray and successive laser removal;
engraved die—the images are engraved directly onto the sides of the stripes in the die, thus creating light/dark effects by means of known removal technologies (such as milling, electrical discharge machining, photo-engraving). In this case, the visual effect is created during the step of forming the cosmetic product directly transferring the image of the die onto the product;
laser—the images are engraved directly through the laser technique.

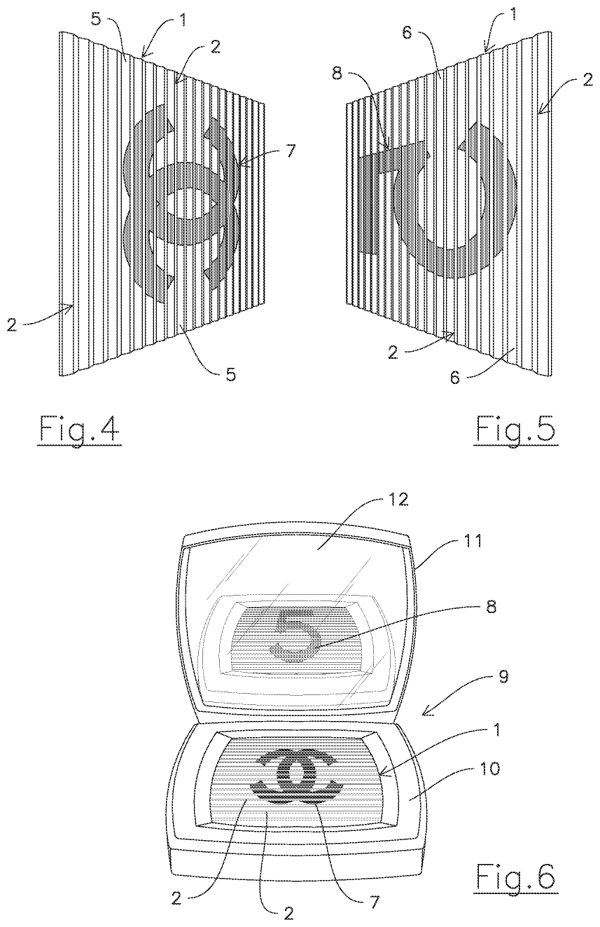

Whichever method is used, due to the inclination of the two sides 5 and 6, looking at the upper surface of the cosmetic plate 1 from opposing observation points A and B angled so as to see only the inclined sides 5 and 6 of the surface (FIG. 2), respectively, it is possible to detect the entire image 7 from point A and the entire image 8 from point B in a separate manner, as shown in FIGS. 4 and 5, respectively.

It results that, by inserting the cosmetic plate 1 into the box-like base 10 of a packaging case 9 with a hinged cover 11 provided with a mirror 12, as shown in FIG. 6, i.e., with the stripes 2 parallel to the rotation axis of the cover 11, it is possible, with the cover open, to observe separately the image 7 inside the base 10 and the image 8 reflected in the mirror 12.

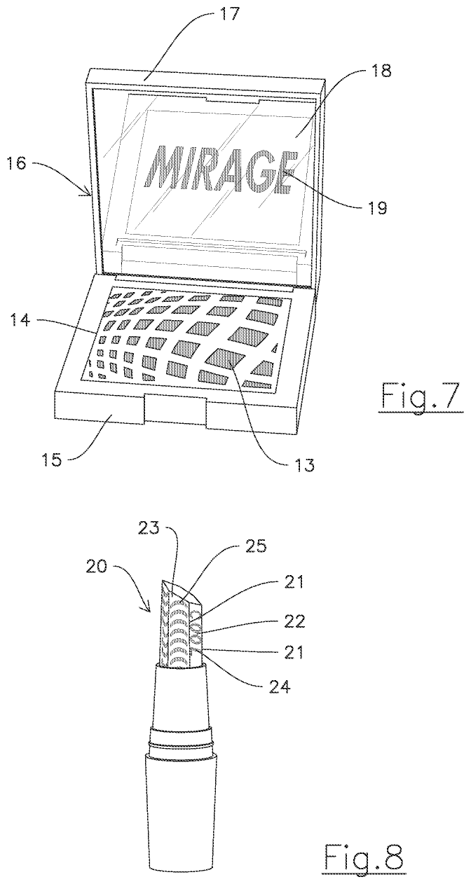

Likewise, it is possible to obtain a package, such as that in FIG. 7, which has a first visible image 13 on the surface of a cosmetic product 14 inserted into the base 15 of a case 16 with hinged cover 17 and mirror 18 and a second image 19 reflected in the mirror 18.

It is also possible to shape the surface of the cosmetic product with stripes not parallel to one another, which are wavy or converging or diverging, whereby the images can also be seen separately by simply rotating the plate or wafer to the right or to the left, not necessarily by using a mirror.

The same inventive principle set forth above for a powder cosmetic product with reference to FIGS. 1-5 and applied, by way of example, to the packages in FIGS. 6 and 7, can be used for decorating a lipstick rod with polygonal section, such as that indicated by reference numeral 20 in FIG. 8. In this case, the vertices of the stripes with triangular section provided by the present invention consist of the corners 21 of the rod 20 and the inclined sides of the stripes consist of the flat faces 22, 23 at the two sides of the corners 21.

The respective images seen from different angles are those indicated by reference numerals 24 and 25.

1-5. (canceled)

6. Cosmetic product made of pressed or melted and baked, or extruded powder, or in the form of a rod or stick, such as a lipstick, with a sight surface provided with a plurality of protruding stripes with triangular section, each stripe having a vertex, a first inclined side decorated with a portion of a first image and a second inclined side decorated with a portion of a second image so that by observing the cosmetic product from an observation point (A) angled to see only the first inclined side of each stripe the first image only is visible, and by observing the same cosmetic product from an observation point (B) angled to see only the second inclined side of each stripe, the second image only is visible.

7. Cosmetic product according to claim 6, wherein said stripes with triangular section have a side inclination from 40° to 60°, a base width from 0.5 mm to 3.0 mm and a height from 0.2 mm to 1.5 mm.

8. Cosmetic product according to claim 6, wherein the cosmetic product is a flat plate or wafer with surface protruding stripes with triangular section.

9. Cosmetic product according to claim 6, wherein the cosmetic product is a lipstick rod with polygonal section.

10. Cosmetic product according to claim 9, wherein said stripes with triangular section are corners of said rod and their inclined sides are flat faces adjacent said corners from opposite parts.
